(19) United States
(12) Patent Application Publication
(10) Pub. No.: US 2016/0123960 A1
(43) Pub. Date: May 5, 2016

(54) METHOD FOR PREPARING THREE-DIMENSIONAL, ORGANOTYPIC CELL CULTURES AND USES THEREOF

(71) Applicant: MILLENNIUM PHARMACEUTICALS, INC., Cambridge, MA (US)

(72) Inventor: Robbie J. ROBERTSON, Somerville, MA (US)

(21) Appl. No.: 14/896,740

(22) PCT Filed: Jun. 10, 2014
(86) PCT No.: PCT/US14/41703

§ 371 Date: Dec. 8, 2015

Related U.S. Application Data
(60) Provisional application No. 61/833,052, filed on Jun. 10, 2013.

(51) Int. Cl. G01N 33/50

(52) U.S. Cl. CPC G01N 33/5023; G01N 33/5088

(57) ABSTRACT

The present invention is directed to methods for generating isolated, unencapsulated, three-dimensional, cell culture products comprising a naturally-derived cell matrix distributed throughout the product and having dimensions suitable for use in in vitro applications including histological applications and imaging.

### REFERENCE TO RELATED APPLICATIONS

This non-provisional patent application claims the benefit of U.S. Provisional Application Ser. No. 61/833,052, filed on Jun. 10, 2013, the contents of which are herein incorporated by reference in its entirety.

### FIELD OF THE INVENTION

The present invention relates to in vitro cell culture. More particularly, the present invention provides a method for preparing isolated, uncapsulated, three-dimensional (3D), organotypic cell culture products.

### BACKGROUND

Predicting response to therapy in cancer patients based on cell line and xenograft studies has been traditionally very difficult. Understanding the effect of pathway perturbation on tumor response in the native tumor microenvironment is of paramount importance. Tumor—stroma interactions have long been recognized as important facets in the pathogenesis and dissemination of malignancy. Significant evidence supporting the role of peritumoral tissues in tumor maintenance includes the presence of genetic mutations in the stroma of several types of cancers (1) and the role played by stromal cells in the acquisition of resistance to therapy (2).

Previous studies that use standard primary cell-culture systems and cell-line s.c. xenografts have advanced our understanding of tumor behavior (3); however, these methods have inherent limitations in evaluating the role of the tumor microenvironment in modulating carcinogenesis and tumor progression. Studies have indicated that cancer cells maintained in vitro in 2D and on plastic under standard tissue culture conditions have adapted to these special environment by changing gene expression patterns and signaling networks (4). Depending on the drug targets studied and the mechanism of action of the compounds analyzed, cells grown under such conditions do not represent an in vivo situation and therefore do not offer a reliable system to test and preselect certain anticancer drugs before testing them in more elaborate in vivo models or in clinical trials. Hence, it is increasingly being recognized that the third dimension and the interaction of different cell types are important in maintaining gene expression patterns and signaling networks operational in vivo and in generating reliable in vitro systems to predict the pharmacological response of therapeutic candidate compounds (5).

Despite the extensive research that has been conducted to date, there is still a need for a simple, inexpensive and flexible method for an improved in vitro culture method which more closely mimics an in vivo environment, is applicable to a wide variety of cells and tissues, can be used with more advanced methods of analysis typically reserved for in vivo models (e.g., imaging), and is suitable for the generation of tens, hundreds or thousands of parallel cultures for high-throughput compound screening. As such, newer, more robust in vitro models need to be developed.

### SUMMARY

The present invention provides an inexpensive, reproducible, rapid, three-dimensional, in vitro cell culture method suitable to investigate epithelial—stromal interactions in tumor onset, progression, and resistance to therapy. Importantly, the methods of the present invention allow for investigation of anti-tumoral pharmacological properties in a system that more closely mimics the original cancer microenvironment. The techniques described herein shed light on the gap that currently exists between results in cell line/xenograft studies traditionally used to predict drug response and actual efficacy in humans.

Surprisingly, cells previously maintained in vitro in 2D adapt and display more organotypic features when cultured in 3D using the methods described herein. For example, analysis of characteristics such as gene expression patterns, signaling networks, response to drug (e.g., sensitivity or resistance) etc., in the three-dimensional, organotypic cell cultures described herein more closely resemble those seen in the tissue sources from which the cells were originally derived as compared to the analysis of the same characteristics of the same cells cultured in 2D.

The invention provides an isolated, unencapsulated, three-dimensional, cell culture product (“OTOC”) comprising a naturally-derived cell matrix distributed throughout the three-dimensional product thus resembling cell-stroma interactions seen in an in vivo microenvironment. The cell culture product of the invention has starting dimensions once created/formed ranging from about 1-10 mm in diameter (length), and about 1-5 mm in width (thickness), and is capable of organotypic growth for several weeks to months. In one embodiment, the cell culture product of the invention has dimensions ranging from about 5-8 mm in diameter (length) and about 3-4.5 mm in width (thickness). In another embodiment, the cell culture product of the invention has dimensions ranging from about 1.5-1.8 mm in diameter (length), and about 1.0-1.4 mm in width (thickness).

In one aspect, the invention provides a method for producing an isolated, unencapsulated, three-dimensional organotypic cell culture product, by performing the following steps: harvesting one or more cells from an in vivo tissue source or from an in vitro culture; resuspending the one or more cells with a naturally derived gel matrix under conditions sufficient to form a liquid cell suspension; directly dispensing at least a portion of the liquid cell suspension into a hydrophobic solution under conditions sufficient to enable the liquid suspension to form a gelled three-dimensional cell matrix (i.e., the organotypic cell culture or “OTOC”) within the hydrophobic solution; isolating the OTOC from the hydrophobic solution; and culturing the OTOC in a growth medium under conditions sufficient for promoting proliferation of the cells within the three-dimensional cell matrix,

In certain embodiments the naturally derived gel matrix is a sol-gel matrix, preferably one having reverse phase characteristics. In other certain embodiments, the naturally derived gel matrix comprises a solubilized basement membrane preparation. The naturally derived gel matrix can further contain one or more matrix proteins such as laminin, collagen IV, heparin sulfate proteoglycans, and enactin, nidogen, or any combination thereof. The naturally derived gel matrix may additionally contain one or more growth factors, including but not limited to TGF-beta, epidermal growth factor (EGF), insulin-like growth factor (IGF-1), fibroblast growth factor (FGF), tissue plasminogen activator,3,4 (tPA), nerve growth factor (NGF), platelet-derived growth factor (PDGF), or any combination thereof. In certain embodiments, the naturally derived gel matrix even further contains heparin sulfate proteoglycan (perlecan) and/or one more matrix metalloproteinases. In a particular embodiment the naturally derived gel matrix is Matrigel (BD Biosciences).

The hydrophobic solution used in the methods of the invention can be an oil, such as mineral oil.

In certain aspects of the invention, the conditions sufficient for forming a liquid suspension involve cooling the gel matrix to approximately 4° C. prior to resuspending the one or more cells in the gel matrix. In contrast, the conditions sufficient to form an unencapsulated, gelled three-dimensional cell matrix (i.e., the OTOC) involve directly dispensing the liquid cell suspension into a hydrophobic solution that has a temperature of 20-25° C.

Preferably, the OTOC is cultured in a three-dimensional culture environment such as a vessel of suitable shape and volume for culturing the OTOC in a sufficient volume of medium. Suitable vessels include a cell culture flask (e.g., T25, T75 or T225 flask) or petri dish of sufficient size/volume, or a multi-well plate (e.g., 6-well, 12-well, 24-well, or 96-well plate).

In another aspect, the invention provides an in vitro method for measuring the pharmacological response to a therapeutic or cytotoxic agent using an unencapsulated, three-dimensional organotypic culture (i.e., OTOC) produced by the methods described above, by the following steps of: contacting the OTOC with a therapeutic or cytotoxic compound; and measuring one or more characteristics of the OTOC subsequent to the contact with the therapeutic or cytotoxic agent. In some embodiments, the therapeutic or cytotoxic agent has detectable label, such as fluorescent label or a radiolabel.

In still another aspect, the invention provides an in vitro method for measuring the response of an unencapsulated, three-dimensional organotypic cell culture (i.e., OTOC) as described above to a modulation of one or more cell culture conditions (e.g., temperature, humidity, % O₂, % CO₂, growth medium, growth medium supplements (e.g., serum), or any combination thereof). A baseline measurement of one or more characteristics of the OTOC is first obtained; one or more culture conditions are modulated for a prolonged period of time (e.g., 2 hours, 3 hours, 4 hours, 6 hours, 12 hours, 24 hours, 48 hours, 72 hours, 84 hours, 96 hours, 108 hours, 120 hours, 144 hours, 168 hours, 10 days, 12 days, or 14 days); then a second measurement of the one or more characteristics of the OTOC is obtained subsequent to modulation of the one or more culture conditions. A change in the second measurement of the one or more characteristics as compared to the baseline measurement(s) is indicative of the response of the OTOC to the environmental condition.

The one or more characteristics being measured can include RNA expression, DNA expression, protein expression, cellular uptake, cellular signaling, cell viability, apoptosis, cell shedding, cellular necrosis, cellular heterogeneity, multicellular interactions, sensitivity or resistance to the therapeutic or cytotoxic agent, or any combination thereof.

In certain embodiments of the methods of the invention, the measuring step involves imaging the unencapsulated, three-dimensional organotypic culture (i.e., OTOC). Suitable imaging methods include but are not limited to optical imaging, nuclear imaging, MRI, SPECT, PET, and Cerenkov Luminescence Imaging (CLI).

In other embodiments of the methods of the invention, the measuring step utilizes an in vitro technique such as immunoflourescence, immunohistochemistry, western blotting, northern blotting and southern blotting, or any combination thereof. Any in vitro assay can also be performed, including but not limited to: a proliferation assay, a cell viability assay, an apoptosis assay, an internalization assay, a cell penetration assay, or any combination thereof.

While the methods described herein are primarily described with respect to tumor cells (e.g., primary or immortalized tumor cells derived from a lung tumor, prostate tumor, breast tumor, ovarian tumor, cervical tumor, colon tumor, gastric tumor, pancreatic tumor, melanoma, lymphoma, or hematologic tumor), any type of mammalian cells can be used with the methods described herein, including but not limited to stem cells, blood cells, immune cells, and/or inflammatory cells. The stem cells can be embryonic stem cells, adult stem cells or induced pluripotent stem cells. Additional types of cells suitable for use in the methods of the invention are described below.

### BRIEF DESCRIPTION OF THE FIGURES

FIG. 1 is a graph depicting the radiance curve from a Celltiter Glow assay using OTOCs generated from HCT-116 (colon tumor) cells and treated with Staurosporine.

FIG. 2 is a graph depicting a growth/viability curve for HCT-116 spheroids (InSphero System) treated with Staurosporine.

FIG. 3 is a line graph depicting ¹⁸F-FDG uptake in OTOCs generated from HT-29 (colon tumor), HCT-116 (colon tumor) and H460 (lung tumor) cells following treatment with a proteasome inhibitor denoted as MLN9708.

FIG. 4 is a line graph depicting ^{18F}-FDG uptake over time in OTOCs generated from SW48 (colon tumor) and SW48 G13D (KRAS mutant) cells and treated with Staurosporine.

FIG. 5 is a graph depicting the radiance curve from a Celltiter Glow assay using OTOCs generated from Calu-6 (lung adenocarcinoma) cells and treated with a UAE inhibitor.

FIG. 6 is a graph depicting the radiance curve from a Celltiter Glow assay using OTOCs generated from Calu-6 (lung adenocarcinoma) cells and treated with a proteosome inhibitor.

FIG. 7 is a graph depicting the radiance curve from a Celltiter Glow assay using OTOCs generated from Calu-6 (lung adenocarcinoma) cells and treated with a VPS34 inhibitor.

FIG. 8 depicts the staining of sagittal sections of HCT-116 OTOCs with Ki67 (100-150 micrometer sections cut every 75 mm).

FIG. 9 a magnified view (20×) of the Ki67 staining of an HCT-116 OTOC depicted in FIG. 8.

FIG. 10 depicts staining of HCT-116 OTOCs with Hif1 alpha.

FIG. 11 is a bar graph depicting ¹⁸F-FDG uptake in H1048 μOTOCs treated for 24 hrs with CoCl₂.

FIG. 12 is a bar graph depicting ¹⁸F-FDG uptake in T47μOTOCs treated for 24 hrs with CoCl₂.

FIG. 13 is a graph depicting the max to min radiance comparison of H1048 and T47D μOTOCs treated for 24 hrs with CoCl₂.

FIG. 14 is a graph depicting the growth/viability curves of OTOCs generated from A549, G13D, SW48, T84, and WSU cells, respectively, over 15 days of growth.

FIG. 15 is a graph depicting the growth/viability curves of OTOCs generated from A549 and T84 cells, respectively, over 35 days of growth.

### DETAILED DESCRIPTION

### Definitions

Throughout the specification, several terms are employed that are defined in the following paragraphs.

The term “2D cell culture”, as used herein, refers to monolayer cultures adherent to rigid substrates.

The term “3D cell culture”, as used herein, refers to any method that includes cell culture in 3 dimensions, with or without the use of a matrix or scaffold.

As used herein, the term “spheroid” refers to either a single cell that divides and grows into a ball, or a forced aggregation of multiple cells, in either case, with or without the use of a matrix or scaffold to support cell growth within the spheroid. The spheroid can be an adherent spheroid or a spheroid grown in suspension (i.e., in liquid or other medium e.g., embedded within a layer of soft agar or Matrigel).

As used herein, the term “hanging droplet” refers to cells that are placed in a hanging drop culture and incubated under physiological conditions until they form spheroids.

As used herein, the term “hanging drop culture” refers to a small drop of liquid, such as plasma or some other media allowing tissue growth, suspended from an inverted rigid substrate. The hanging drop is then suspended by gravity and surface tension, rather than spreading across a plate. This allows tissues or other cell types to be examined without being squashed against a dish.

By “organotypic culture” is meant that the cells associate in a way that as closely as possible replicates the biochemical and physiological properties (e.g., cell-stroma interactions) of the organ or tissue source (e.g., a solid or a hematologic tumor) from which the cells are derived. Preferably the cells are dissociated cells, microexplants or explants.

As used herein, the term “dissociated cell(s)” refers to a cell(s) that has been isolated from an organ or a tissue source (e.g., a solid or a hematologic tumor).

As used herein, the term “hydrogel” has its art understood meaning and refers to a water-swellable polymeric matrix that can absorb water to form gels of varying elasticity. On placement in an aqueous environment, dry hydrogels swell to the extent allowed by the degree of cross-linking. The amount of water absorbed can be controlled by the macro molecule component used. A hydrogel can enclose or comprise a pharmaceutically active agent and/or a biologically active agent.

The term “matrix” or “extracellular matrix” refers to a 3D network of macromolecules held together by covalent and/or non-covalent crosslinks. Other terms are defined in the following section when needed.

### 3D Cell Culture

Cell-based assays have become an integral component in many stages of routine anti-tumor drug testing. However, they are almost always based on a 2D homogenous monolayer or suspension cultures and thus represent a rather artificial cellular environment. In contrast, an in vivo mature tumor with an extensive vasculature has a very complex structure, consisting of regions of regularly dividing cells, hypoxic cells and necrosis zones, at increasing distances from blood vessels with 3D pattern bearing structural heterogeneity.

Studies have shown that data obtained for novel therapeutic agents in established cell lines grown adherently in 2D on plastic should be interpreted with great caution. It is well known in the literature that in a 2D in vitro environment, cell phenotypically differ from that of the in vivo setting. Expression levels, migration ability and changes in apoptosis are just of few of the well documented differences noted between an in vitro and an in vivo growth environment (21). Spatial and temporal organization of cells in a 3D environment plays a critical role in proper cell cycle progression, drug resistance and stress response. It can also provide a more accurate indication of in vitro to in vivo translation with greater drug predictability than current 2D cell cultures.

Several 3D cell culture methods have been exploited in the past to culture cells (e.g., cancer cells and tumor tissue) from patients. For example, organotypic cultures such as slice cultures have been characterized in terms of tissue integrity, cell viability, and gene as well as signaling pathway expression. Multicellular tumor spheroids, first developed in the 1970s (6), were designed to mimic the 3D-structure of small solid tumors, representing small tumor nodules with nutrient, pH, and oxygen gradients as well as cells at different physiological states depending on their location in the 3D organoid (17). Several different spheroid systems have been developed, including spheroids grown as aggregates: (a) on gels of the basement membrane-like Matrigel, (b) on plastic coated with poly-HEMA, (c) on SciVax nanoculture plates, (d) as aggregates in suspension culture, or (e) as aggregates using the InSphero hanging droplet system,. The spheroids resulting from these 3D culture systems are all multicellular, multilayered aggregates that lack a matrix within the aggregated structure to support cell growth. Additionally, some spheroid techniques previously have involved mixing tumor cells with fibroblasts in spheroids (7). While such 3D cell cultures systems are believed to better reflect the in vivo behavior of cells in tumor tissues, it remains open, so far, whether stimulated expression of differentiation markers is caused by enhanced cell-to-cell communication or is displayed only by the cells in direct contact with the gels or other growth substrates used.

Cell encapsulation methods (e.g., using PEG, agarose or alginate as encapsulation agents) have been proposed as a technique to quickly and easily prepare a large number of spheroids with size distributions within desired diameter ranges (10, 11). The idea of tumor cell encapsulation for generating tumor spheroids was developed and extended to testing chemotherapeutical drugs (8, 9). However, studies to date have shown that even in these systems, the complexity of the tumor microenvironment is poorly preserved, and most tumor cell—stroma interactions are missing.

Additionally, classical in vitro methods used to create traditional spheroids, such as liquid-overlay, spinner flask and gyratory rotation systems, are time consuming and cannot provide the production of spheroids with desired size ranges. Moreover, some tumor cell types are alone incapable of forming spheroids at all.

The invention provides cell culture methods for producing isolated, unencapsulated, 3D, organotypic cell products (“OTOCs”). OTOCs are produced by combining a naturally derived matrix with reverse phase characteristics (i.e., remains in liquid form when chilled, and begins the gelling process as it warms to room temperature) with one or more desired cells directly obtained from an in vivo tissue source, or obtained from a traditional 2D in vitro cell culture (e.g., primary or immortalized cells) to form a cell slurry or suspension. Suitable matrices for use in the invention are described below. The cell slurry/suspension is then pipetted directly into a hydrophobic solution, such as an oil, and allowed to gel. Because the cell slurry/suspension is hydrophilic and the oil is hydrophobic, the hydrophilic component naturally forms a ball or spherical structure within the oil as it comes to room temperature. The resulting unencapsulated, 3D, organotypic cell product (OTOC) is then removed from the hydrophobic solution, rinsed and placed in a three-dimensional cell culture environment (e.g., in a flask or a petri-dish containing a suitable volume of growth medium to completely surround the OTOC on all sides) under conditions sufficient to promote proliferation of the cells for days, weeks or months until the desired three-dimensional size is achieved. The cell culture is conducted using “appropriate culture medium and conditions”. The term “appropriate culture medium and conditions” refers to a culture medium and to environmental conditions (including but not limited to temperature, humidity % O₂ and % CO₂), that support survival and proliferation of cells cultured in a matrix. Such cell culture media and conditions are known in the art.

The methods of the present invention provide a valuable tool to profile compounds in a 3D, organotypic cell culture system (referred to herein as “OTOCs”) that is closer, and thus is expected to be more predictive, to the clinical situation than prior 3D cell culture methods described in the art. The methods of the invention offer a unique, simple and inexpensive 3D system to test and select the best drugs and drug combination therapies in vitro prior to performing animal experiments.

Unlike the prior art methods for producing 3D cell cultures, the methods of the invention do not require co-culture with fibroblasts (7), nor do they require entrapment or encapsulation of the cells to control or restrict cell growth (8, 9). The use of a naturally derived matrix to in forming the OTOCs thereby having a distributed matrix throughout the OTOC to support cell growth, provides the cell-stroma interactions that prior 3D spheroid systems lack. As such, the OTOCs of the invention display more organotypic growth features as seen in vivo as compared to prior 3D spheroids described in the art.

The starting size of OTOCs once seeded/formed range from about 1-10 mm in diameter, and about 1-5 mm in width and can be grown for several weeks to months while still maintaining organotypic growth features. 3D cell cultures produced by most prior art methods are capable of growth for typically only 3-5 days before developing necrotic regions or cores that lead to death of the 3D culture. In one embodiment, the OTOCs of the invention have an average starting size ranging from about 5-8 mm long (diameter), and about 3-4.5 mm wide (thick). In another embodiment, the average starting size ranges from about 1.5-1 8 mm long (diameter), and about 1.0-1.4 mm wide (thick) (referred to herein as μOTOCs).

The starting sizes of OTOCs and μOTOCs and their capability for continued growth over an extended period of time ranging from weeks to months make them suitable for use in a variety of in vitro applications, including but not limited to viability assays, histological applications, and imaging. The starting sizes of μOTOCs also makes them suitable for use in 96 well format to enable their use in high throughput assays and other high throughput screening methods. Other 3D cell culture methods, such as traditional spheroids or hanging droplets, are too small to use in imaging applications and are incapable of growing to sizes comparable to OTOCs due to inherent size limitations in their growth. More specifically, traditional 3D cell cultures like spheroids or hanging droplets typically require encapsulation and/or do not utilize an extracellular matrix within the spheroid to support three dimensional growth. As such, the cells layer directly on top of one another as they grow, forming an aggregated structure that eventually results in necrotic regions once they reach a size of approximately around 100-500 μm in size.

In contrast to prior methods of 3D cell culture in which perfusion of oxygen and nutrients from growth medium is typically two-dimensional (e.g., from a tube or other media source from above, as in a hanging droplet system, or a tube or other media source from below, as in an adherent spheroid system), the methods of the invention allow for 360° perfusion of oxygen and nutrients, such that perfusion occurs on all sides of the 3D cell product formed by the methods described herein. As such, the methods of the invention provide a truly 3D cell culture system which utilizes a three-dimensional culture environment to promote three-dimensional cell growth, thus more closely mimicking growth in an in vivo microenvironment.

Table 1 below summarizes some of the primary the advantages of the 3D, organotypic cell culture system of the invention as compared to 3D cell culture systems previously described in the art.

TABLE 1
Advantages of OTOCs over other 3D cell culture systems

| Type of 3D | | | | |
|---|---|---|---|---|
| cell culture | | | | Advantages of |
| System | Size | Benefits | Limitations | OTOCs |
| Spheroid | 100-500 | High | Inherent size limitations; | Larger size; suitable |
| culture | μm | throughput, | not suitable for imaging or | for imaging and |
| | | ease of use | histological use; no | histological use; ECM |
| | | | extracellular matrix (ECM) | within OTOC |
| | | | within the spheroid | supports organotypic |
| | | | structure to support | growth; no |
| | | | organotypic growth; | encapsulation required |
| | | | encapsulation sometimes | |
| | | | required | |
| Hanging | 100-500 | High | Inherent size limitations; | Larger size; suitable |
| droplets | μm | throughput, | not suitable for imaging or | for imaging and |
| | | ease of use | histological use; no | histological use; ECM |
| | | | extracellular matrix (ECM) | supports organotypic |
| | | | to support organotypic | growth |
| | | | growth; | |
| 3D Matrices | Variable | Uniform, | Expensive; typically | Ease of preparation; |
| or | | tightly formed | synthetic; Uniform | ECM supports |
| Scaffolding | | environment | environment is not | organotypic growth |
| | | | representative of in vivo | (more closely |
| | | | growth conditions | resembles true in vivo |
| | | | | environment) |
| Tissue | Thin | Live Tissue | Need healthy, sterile tissue, | Requires cells only; |
| Sections | sections | | short lived | cultures survive and |
| (slice | (several | | | maintain organotypic |
| culture) | μm) | | | features for weeks to |
| | | | | months |

The invention thus provides a simple method of generating unencapsulated, 3D, organotypic cultures using cells, including primary or immortalized cells from a wide variety of tumors (and other tissue sources as described herein) that can be maintained for several days, weeks or even months while maintaining organotypic characteristics, and have sufficient sizes suitable for use in a variety of in vitro applications including histology and imaging. The fact that the 3D cell cultures described herein are easy to produce and maintain also makes them ideal for the construction of many thousands of parallel cultures for high throughput screening of drug candidates. Furthermore, the cells themselves can be genetically manipulated efficiently prior to setting up the 3D organotypic culture, introducing one or more transgenes by means of transfection or transduction in an appropriate vector, or by introducing siRNA as oligonucleotide or expressed from a suitable vector.

### 3D Cell Matrices and Scaffolds

The majority of cell culture studies have been performed on two-dimensional (2D) surfaces such as micro-well plates, tissue culture flasks, and Petri dishes because of the ease, convenience, and high cell viability of 2D culture. Although these conventional 2D cell culture systems have tremendously improved our understanding of basic cell biology, they have proved to be insufficient and unsuitable for new challenges in cell biology as well as for pharmaceutical assays. For example, the chemically and spatially defined three-dimensional network of extracellular matrix components, cell-to-cell and cell-to matrix interactions that governs differentiation, proliferation and function of cells in vivo is, in fact, lost under the simplified 2D condition. Indeed, 2D culture systems fall short of reproducing the complex and dynamic environments of the in vivo situation, which are known to affect cell morphology, growth rates, contact geometries, transport properties, and numerous other cellular functions.

3D cell culture represents a potential bridge to cover the gap between animal models and human studies, being able to reproduce specific tissue-like structures and to mimic functions and responses of real tissues in a way that is more physiologically relevant than what can be achieved through traditional 2D cell monolayers. 3D in vitro systems can improve the predictive value of cell-based assays for safety and risk assessment studies and for the development and testing of new drugs.

Three-dimensional (3D) cell culture matrices, also called scaffolds, have been introduced to overcome 2D cell culture limitations. These matrices are porous substrates that can support cell growth, organization, and differentiation on or within their structure. Architectural and material diversity is much greater on 3D matrices than on 2D substrates. Table 2 summarizes the function of extracellular matrix in native tissues and the analogous function of 3D cell matrices/scaffolds in engineered tissues.

TABLE 2

| | | Analogous functions of | |
|---|---|---|---|
| | Functions of ECM in native | scaffolds in engineered | Architectural, biological, and |
| | tissues | tissues | mechanical features of scaffolds |
| 1. | Provides structural support for | Provides structural support | Biomaterials with binding sites for cells; |
| | cells to reside | for exogenously applied cells | porous structure with interconnectivity for |
| | | to attach, grow, migrate and | cell migration and for nutrients diffusion; |
| | | differentiate in vitro and in | temporary resistance to biodegradation |
| | | vivo | upon implantation |
| 2. | Contributes to the mechanical | Provides the shape and | Biomaterials with sufficient mechanical |
| | properties of tissues | mechanical stability to the | properties filling up the void space of the |
| | | tissue defect and gives the | defect and simulating that of the native |
| | | rigidity and stiffness to the | tissue |
| | | engineered tissues | |
| 3. | Provides bioactive cues for cells | Interacts with cells actively | Biological cues such as cell-adhesive |
| | to respond to their | to facilitate activities such as | binding sites; physical cues such as surface |
| | microenvironment | proliferation and | topography |
| | | differentiation | |
| 4. | Acts as the reservoirs of growth | Serves as delivery vehicle | Microstructures and other matrix factors |
| | factors and potentiates their | and reservoir for exogenously | retaining bioactive agents in scaffold |
| | actions | applied growth-stimulating | |
| | | factors | |
| 5. | Provides a flexible physical | Provides a void volume for | Porous microstructures for nutrients and |
| | environment to allow | vascularization and new | metabolites diffusion; matrix design with |
| | remodeling in response to tissue | tissue formation during | controllable degradation mechanisms and |
| | dynamic processes such as | remodeling | rates; biomaterials and their degraded |
| | wound healing | | products with acceptable tissue |
| | | | compatibility |

A variety of biomaterials and fabrication processes have been developed or adapted to produce 3D cellular supports (also referred to as 3D matrices or scaffolds) with different physical appearance, porosity, permeability, mechanical characteristics, and nano-scale surface morphology in an attempt to match the diversity of in vivo environments. Examples of such materials include, for example: collagen gels, sponges or biogels; fibrin; fibronectin; laminin; alginates, hydrogels and composites; cross-linked glycosaminoglyca; silk composites; PGA, PLLA, PEG and other polymer-based, synthetic scaffolds; nanofibers and peptide scaffolds; and chitosan composites. The prior art teaches away from the use of naturally derived matrices or scaffold materials in 3D cell culture due to perceived disadvantages associated with batch-to-batch variability, contamination from growth factors presenting known and unknown experimental confounders. While some natural materials, such as collagen and fibrin, are more standardized, having little batch-to-batch variability the use of Matrigel in 3D cell culture was particularly avoided in the art due to its known batch-to-batch variability and the high degree of contamination from known and unknown growth factors, in addition to its weak mechanical strength, and the inability to adjust its matrix mechanics due to resulting confounding alterations in matrix biochemistry (23). Synthetic scaffold materials are very uniform in nature, and thus create a very tightly controlled growth environment. The use of such controlled or uniform scaffolding material is believed by many skilled in the art to facilitate direct comparisons between experiments.

The present invention is based, in part, on the discovery that uniformity and tight control within the growth environment of prior 3D cell culture methods is not representative of the dynamic nature of an in vivo growth environment, and that cells grown using such uniform scaffolding materials do not behave as they would in an actual in vivo setting. Despite the teachings of the prior art against the use of naturally derived matrices or scaffold materials, the methods of the present invention provide for the use of naturally derived matrices having inherent variability within its matrix biochemistry and batch-to-batch variability, thereby loosening the restrictions under which the cells grow and exposing them to the same type of variability experienced in a typical in vivo setting.

Preferably, the naturally derived 3D matrix or scaffold used in the methods of the invention is a gel matrix. Preferably the gel matrix is a sol-gel matrix with reverse phase characteristics (i.e., liquid phase when chilled (e.g., 4° C.), solidifies into a gel at room temperature (e.g., 20-25° C.)). In some aspects, the 3D matrix/scaffold include one or more growth factors, including but not limited to TGF-beta, epidermal growth factor, insulin-like growth factor, fibroblast growth factor, nerve growth factor, platelet derived growth factor, and tissue plasminogen activator,3,4, or any combination thereof.

In certain aspects, the 3D matrix used in the methods of the invention is a crosslinked hyaluronan hydrogel as described in Published PCT Application No. W02011161172, the contents of which are hereby incorporated by reference in its entirety.

In a particular embodiment, the gel matrix used in the methods of the invention is a basement membrane matrix preparation such as MatrigelTM (BD Biosciences).

### Cell Types for OTOC Generation

While the methods described herein are primarily described with respect tumor cells (e.g., from primary or immortalized lung tumor, prostate tumor, breast tumor, ovarian tumor, cervical tumor, colon tumor, gastric tumor, pancreatic tumor, melanoma, lymphoma, or hematologic tumor) any type of mammalian cells can be used with the methods of the invention.

Additional types of cells that can be cultured using the 3D cell culture methods described herein include stem cells, induced pluripotent stem cells, progenitor cells, and differentiated cells. Suitable cells may be of a single cell types (e.g., cardiomyocytes or fibroblasts) or may comprise at least two different cell types {e.g., keratinocyte-fibroblast co-culture). Preferably, cells to be cultured on a crosslinked hyaluronan hydrogel of the present invention are of mammalian (animal or human) origin. Mammalian cells may be of any organ, fluid or tissue origin {e.g., brain, liver, skin, lung, kidney, heart, muscle, bone, bone marrow, blood, amniotic fluid, umbilical cord blood, etc) and of any cell type (see below). Cells may be primary cells, secondary cells or immortalized cells {i.e., established cell lines). They may be isolated or derived from ex vivo biological samples or obtained from volunteers or patients by techniques well known in the art, or alternatively they may be purchased from commercial resources (for example, from the American Type Culture Collection, Manssas, VA). Alternatively or additionally, cells may be engineered to contain a gene of interest such as a gene expressing a growth factor or a receptor, or to contain a defective gene, or yet to contain Oct3/4, Sox2, Klf4, and c-Myc genes in order to prepare human induced stem cells from adult somatic cells.

Examples of adult differentiated cells that can be grown in a 3D according to the methods of the present invention include, but are not limited to, basal cells, epithelial cells, platelets, lymphocytes, T-cells, B-cells, natural killer cells, reticulocytes, granulocytes, monocytes, mast cells, neurocytes, neuroblasts, glioblastom, cytomegalic cells, dendritic cells, macrophages, blastomeres, endothelial cells, interstitial cells, Kupffer cells, Langerhans cells, littoral cells, tissue cells such as muscle cells and adipose cells, osteoblasts, fibroblasts, and the like.

Examples of progenitor cells that can be grown according to the methods of the present invention include, but are not limited to, hematopoietic progenitor cells, endothelial progenitor cells, neural progenitor cells, mesenchymal progenitor cells, osteogenic progenitor cells, stromal progenitor cells, and the like.

Examples of stem cells that can be grown in 3D using the methods of the present invention include, but are not limited to, embryonic stem cells, adult stem cells and induced pluripotent stem cells.

In vitro Analysis Using the OTOC System

The 3D, organotypic cell culture system (OTOC) of the invention has broad utility for both research purposes and commercial applications. For example, response readouts can be performed via western, northern or southern blot, and/or histological techniques (e.g., immunohistrochemistry, in situ hybridization, immunoflourescence). More importantly, the 3D cell culture systems of the invention can also be utilized for investigating/monitoring environmental and pharmacological response of cells (e.g., tumor cells) in vitro using current nuclear/optical imaging methods such as MRI, SPECT, positron emission topography (PET) or Cerenkov luminescence imaging (CLI) techniques, etc. In contrast, the 3D cell culture systems previously described in the art have not been suitable for use in an imaging setting (e.g., due to the inherent size limitations of spheroids of hanging droplets).

Additionally, the OTOC system described herein is useful for performing in vitro experiments for cancer metastasis, high throughput drug selection, radioprobe development for cells and tissues, stem cell research, investigation of multicelluilar interactions and tissue heterogeneity, chemotaxis, antibody penetration rates, internalization assay for radioisotope labeled compounds, antiproliferation assays, and spectroscopic analysis.

Other examples of applications involving the OTOC system of the invention include, but are not limited to, proliferation of cells and tissues in vitro in an environment that more closely approximates that found in vivo (for example as research tools), screening of pharmaceutical compounds and toxicology assays in such cell cultures or tissues in vitro, cell therapy, cell delivery, drug delivery, biochemical replacement, production of biologically active molecules, tissue engineering {e.g., ex vivo organ model, tissue explants, in vivo tissue regeneration), biomaterial, and clinical trials.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Generation of Isolated, Unencapsulated, 3D, Organotypic Cell Culture Products (OTOCs and μOTOCs)

Unencapsulated, 3D, organotypic cell culture products (referred to herein as “OTOCs” and μOTOCs) were created utilizing the difference between the hydrophilic and hydrophobic characteristics of a reverse phase gel matrix and oil, respectively, using the following materials and methods.

Materials: Cell culture incubator set to 37° C. (95% O₂, 5% CO₂); pipette tips, test tube rack, autoclaved mineral oil (Sigma Aldrich Cat. # M5904-5X5ML); 10 sterile 5 ml polystyrene test tubes; MatrigelTM (BD Biosciences); T75 cell culture flask; cell strainer (40 μm nylon (BD Falcon Cat. #1119020).

Methods: Approximately 1×10⁴ to 1×10⁶ cells (subject to change depending on the size and types of cells and their respective growth rates) are harvested from culture plates or flasks, counted, spun down into a pellet, and resuspended in cold (4° C.) 100% Matrigel. This Matrigel/cell mixture is pipetted below the level of warmed (37° C.) mineral oil. A “Matrigel/cell ball” forms due to the interaction of the oil/water difference which prevents the Matrigel and oil from mixing. The temperature of the warmed oil facilitates the solidification of the Matrigel/cell mixture into a gel matrix. The OTOCs are then removed from the oil, rinsed and grown under standard cell culture conditions (37° C. (95% O₂, 5% CO₂ in a water jacketed cell incubator). Cells can be grown in either cell culture flasks or in a petri dish environment.

### Procedure:

In a hood, 1 ml of sterile mineral oil was added to each 5 ml polystyrene test tube. Tube tops were recapped and the test tube rack was placed for ˜15-20 in a 37° C. cell culture incubator (95% 02, 5% CO2) prior to use. The desired cells were harvested from 2D culture, counted and spun down into a pellet. Supernatant was removed leaving the cell pellet. Enough cold (4° C.) Matrigel was added to the cell pellet to provide a 1×10⁵ cells/100 μl suspension. The cell/Matrigel solution was mixed thoroughly with a pipette and placed on ice. 100 μl of the Matrigel/cell suspension was drawn into a pipette tip, and the tip of the pipette was then placed just below the surface of the warmed (37° C.) mineral oil and the cell solution was delivered into the oil. The tip of the pipette was removed from the oil and the resulting cell ball settled on the bottom of the tube. Additional 100 aliquots of the Matrigel/cell suspension was delivered into the warmed mineral oil until the desired number of OTOCs were formed. Each OTOC measured approximately 5-8 mm long (diameter); 3-4.5 mm wide (thick). The tube with the mineral oil and newly created OTOCs was placed in a 37° C. incubator for 15-20 minutes. The OTOCs were then strained from the oil with a cell strainer in a 50 ml conical tube (inverting the tube for a few seconds facilitated release of the OTOC from the bottom of the tube). The OTOCs were then rinsed 3 times with 30 ml of 37° C. sterile PBS then poured from the strainer basket into a T75 flask filled with 35 ml of appropriate media for the chosen cell line.

The procedure above can be modified to generate OTOCs suitable for use in 96-well plate format (referred to as μOTOCs) by resuspending the harvested cell pellet with enough cold (4° C.) Matrigel to provide a 1×10⁵ cells/5 μl, suspension, and drawing 5 μL of the Matrigel/cell suspension into a pipette tip, placing the pipette tip just below the surface of warmed (37° C.) mineral oil and delivering the cell solution into the oil. Additional aliquots of the 5 μL of the Matrigel/cell suspension can be delivered into the warmed mineral oil until the desired number of μOTOCs are formed. The resulting cell balls (i.e., μOTOCs) are approximately 1.5-1.8 mm long (diameter) and 1.0-1.4 mm wide (thick) and settle on the bottom of the tube. The μOTOCs are then isolated by cell strainer or pipette, rinsed 3 times with 30 ml of 37° C. sterile PBS and placed into a T75 flask and cultured for 3 days. On Day 3, a pipette tip is then used to transfer the μOTOCs from the T75 flask to a 96 well plate (1 μOTOC/well) for high throughput assaying.

Table 4 summarizes OTOCs that have been successfully generated from a variety of tumor cell lines using the materials and methods described above.

TABLE 4

| Tumor Cell Line | Tumor Cell Type |
|---|---|
| OCI-Ly10 | Lymphoma |
| MDA-MB-231 | Breast |
| CWR22RV1 | Prostate |
| HT-29 | Colon |
| HeLa | Colon |
| Caki-1 | Kidney |
| H460 | Lung |
| HCT-116 | Colon |
| U87MG | Brain |
| Calu-6 | Lung |
| A549 | Lung |
| T84 | Colon |
| SW48 | Colon |
| SW48 G13D | Colon |
| WSU-DLCL2 | Lymphoma |
| PHTX-9C-Luc | Colon |
| T47D | Mammary gland |
| | (ductal carcinoma) |
| H1048 | lung |

Typically, a decrease in cell uptake of 18F-FDG is seen after initial creation of the OTOC. This viability continues to decrease over the next 3-7 days (depending on the cell line used), at which time the cells stabilize and begin to grow. 18F-glucose avidity typically increases around this time frame (see e.g., Example 3).

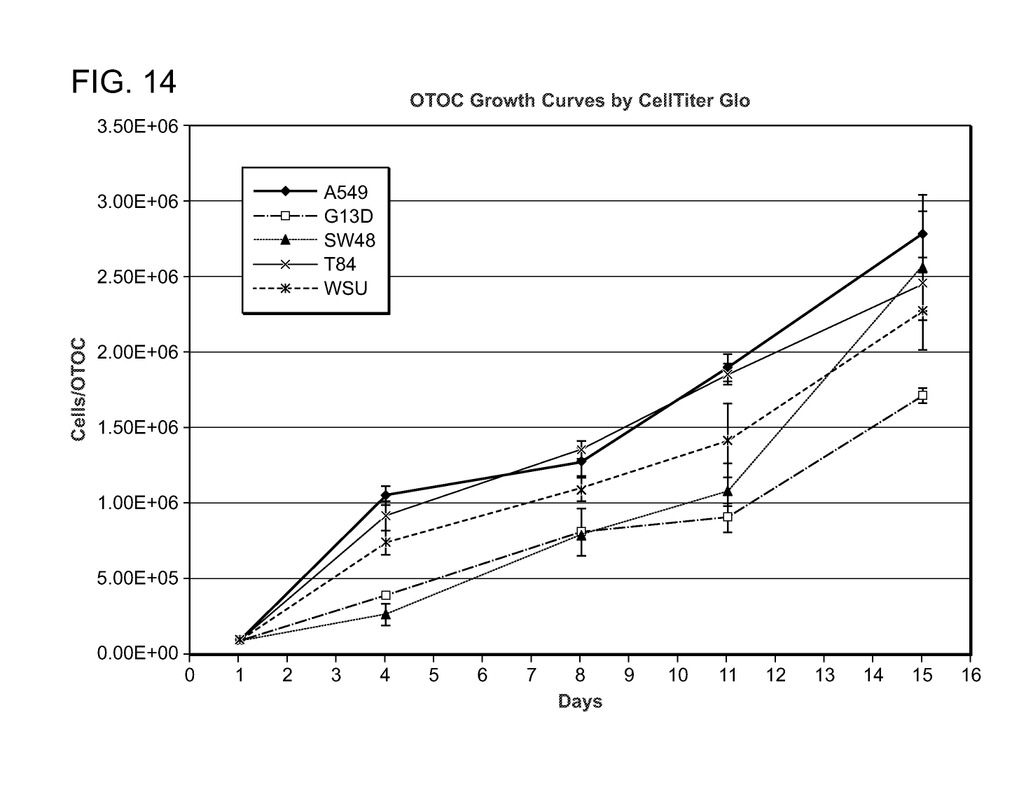

The growth/viability of OTOCs generated from A549, G13D, SW48, T84, and WSU cells, respectively, was measured over 15 days of growth using a Cell Titer Glo assay. The OTOCs were generated using the methods described above. Measurements were taken on Day 1 post-seeding/creation of the OTOCs, and then again on Days 4, 8, 11 and 15. The OTOCs were lysed in Cell Titer Glo by placing each OTOC in separate glass test tubes with several glass beads. Approximately 800 μl of PBS was added to each test tube and the tube was vortexed for 2-3 min to disrupt the OTOC. After vortexing each test tube was placed on ice until sampling. A black 96 well plate was used for the samples. For each lane 100 μl of the OTOC solution was placed in each well (8 wells/800 μl (total)/OTOC) until all OTOCs samples were completed. 100 μl of Celltiter glow was added to each well and the plates were rested for 10 min before reading. IVIS Spectrum was run on Auto exposure for each plate and the Living Image analysis program was used to determine Radiance/per well. Lanes were added up and group average and error was determined. As shown in FIG. 14, OTOCs generated from each of the tumor lines tested exhibited continuous growth over the course of 15 days.

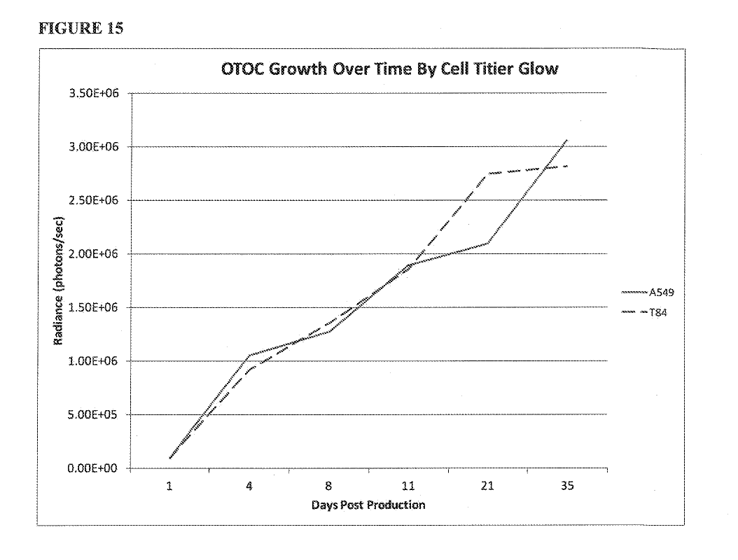

In another separate study, the growth/viability of OTOCs generated from A549 and T84 cells, respectively, was measured over 35 days of growth using a Cell Titer Glo assay. The OTOCs were generated using the methods described above. Measurements were taken on Day 1 post-seeding/creation of the OTOCs, and then again on Days 4, 8, 11, 21 and 35. As shown in FIG. 15, OTOCs generated from A549 and T84 cells exhibited continuous growth over the course of 35 days.

As demonstrated herein, OTOCs are suitable for use in in vitro assays and other methods of in vitro analysis (e.g., western blot, northern blot, southern blot, histological applications, imaging applications) within just a few days of production.

### Example 2

### HCT-116 OTOCs-IC50 Testing with Staurosporine (STP)

The objectives of this study was to determine the IC50 for Staurosporine (“STP”) in the OTOCs assay using the colon cancer cell line HCT-116 (HCT-116-Luc), and to compare the level of sensitivity between a spheroid culture assay and the OTOC assay using the HCT-116 cell line in each assay.

A total of 40 HCT-116 OTOCs were generated in a T225 flask housing using the methods described in Example 1. 24 OTOCs were removed and placed in a 6-well plate, 4 OTOCs/well on day 3 post OTOC seeding/creation.

Staurosporine (Sigma Cat#S5921-1MG) was diluted as follows (FW: 446.53 g/mol Lot# 098K4000):

1 mg/8.9 ml of media is 250 μM stock

1 ml of stock: 9 ml media is 25 μM

1.08 ml of 25 μMstock: 8.92 ml medium is 2.7 μM

120 μl of 25 μMstock: 9.88 ml medium is 0.3 μ

13.6 μl of 25 μMstock: 9.98 ml medium is 0.034 μM

1.6 μl of 25 μMstock: 9.99 ml medium is 0.004 μM

Each well in the 6-well plate (4 OTOCs/well) received a different staurosporine treatment (Control (media only), 25 uM, 2.7 uM, 0.3 uM, 0.034 uM, and 0.004 uM). At 4 days post drug administration all OTOC's were collected for viability measurement to look at growth kinetics and drug resistance to STP.

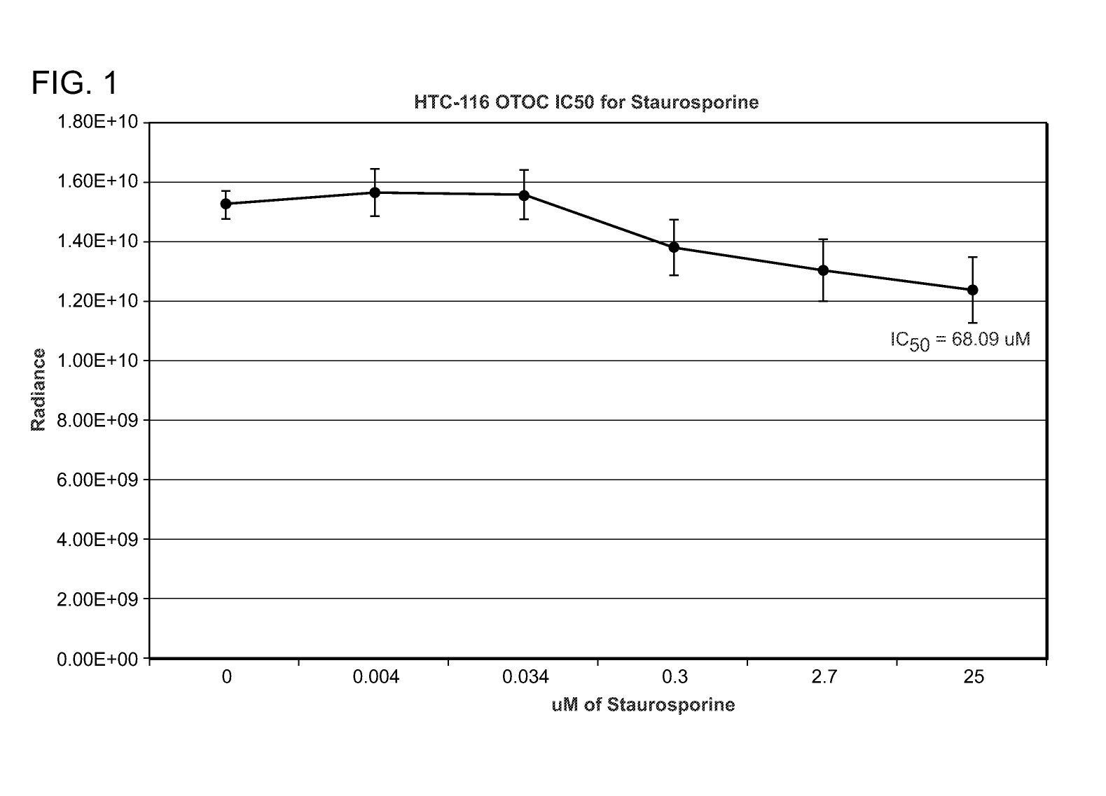

Each OTOC was placed in a glass test tube with several glass beads. Approximately 800 of PBS was added to test tube and the tube was vortexed for 2-3 min to disrupt the OTOC. After vortexing each test tube was placed on ice until sampling. A black 96 well plate was used for the samples. For each lane 100 μl of the OTOC solution was placed in each well (8 wells/800 (total)/OTOC) until all OTOCs samples were completed. 100 μl of Celltiter glow was added to each well and the plates were rested for 10 min before reading. IVIS Spectrum was run on Auto exposure for each plate and the Living Image analysis program was used to determine Radiance/per well. Lanes were added up and group average and error was determined. The results are shown in FIG. 1.

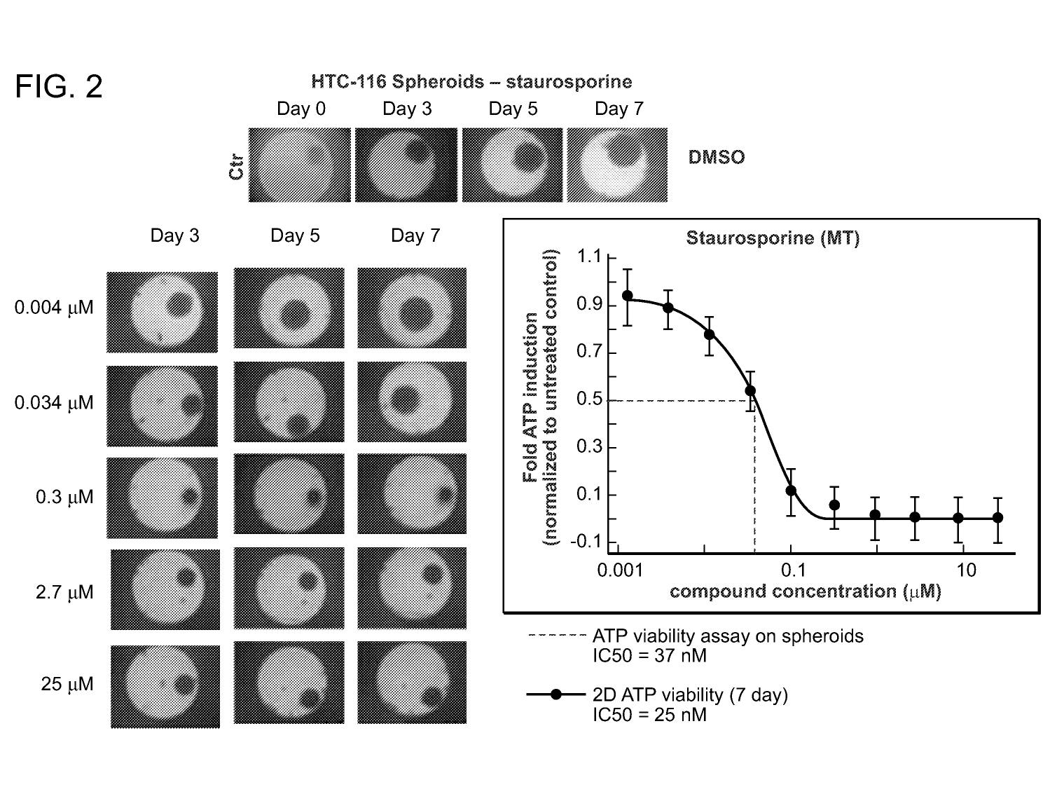

As described above, the HCT-116-Luc cell line was used in an OTOC assay to determine the IC₅₀ of the HCT-116 cell line in the context of the OTOC format following addition of Staurosporine (STP). In a previous InSphero assay (hanging drop spheroids generated in house), an IC₅₀ of 37 nM was established with this cell line (FIG. 2). This experiment that was undertaken was to determine if there was an increase in the resistance of known drug in comparison of the spheroid culture to OTOC format. The same conditions that were use in the previous InSphero experiment (in regards to dose and timing) were used in this experiment. OTOC's were created and on Day 3 (post creation) they underwent treatment with STP. Day 4 post Celltiter Glow assay for this cell line gave a 68 μM IC₅₀. Without intending to be bound by any theory, the differences in IC₅₀ could be the result of the OTOC assay format creating a greater resistance phenotype. It appears that the OTOC assay produces a noticeable difference from that of the InSphero assay with this cell line. The, ratio of the two IC₅₀s (InSphero vs. OTOC) suggests that there is an 1800× greater resistance in this experiment to that of the InSphero spheroid assay with STP.

The study described above was repeated using newly generated HCT-116 OTOCs and InSphero spheroids, and the findings were confirmed. Again, there was an 1800× greater resistance to STP in the OTOCs assay as compared to the InSphero assay in this repeated experiment.

### Example 3

### Imaging of OTOCs

The objective of this study was to monitor radio-labeled compound uptake in vitro using the OTOC system described herein. Three tumor cell lines (HT-29 (colon tumor cell line), HCT-116 (colon tumor cell line), and H460 (lung tumor cell line) were used to generate OTOCs in order to test the effects of FDG uptake after drug treatment with MLN9708, a proteasome inhibitor. OTOCs for each cell line were transferred to 6 well plates, respectively, with 6 OTOCs/well.

An in vitro dosage of MLN9708 was extrapolated from historical in vivo PK studies for this compound. A 700 ng/ml concentration was decided upon and used in the OTOC studies described herein (derived from the mean tumor concentration achievable with a standard dose of MLN9708 (11 mg/kg PK dose, IV)).

The imaging modality Cerenkov Luminescence Imaging (CLI, Robertson et al. (2009) Phys. Med. Biol. 54:N355-N365) was used to monitor the FDG response in vitro.

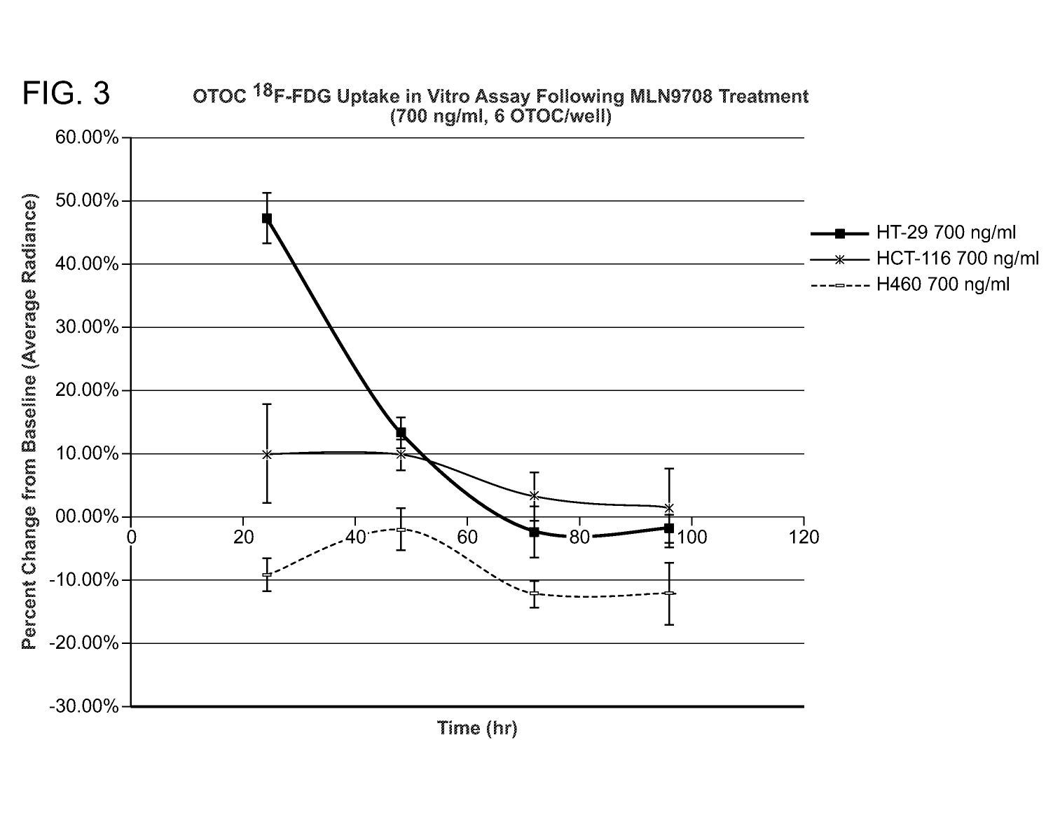

The cell line HT-29 (KRAS WT) is known to be responsive to MLN9708 in vivo (t/c <0.3), while HCT-116 and H460 (both KRAS mutants) have shown resistance to proteasome inhibition (data not shown). OTOCs were imaged via CLI at 24, 48, 72, 96 hours post compound administration. The data in FIG. 3 shows a difference in FDG uptake values between the responsive and non-responsive lines. HT-29 had an increased uptake at the 24 hr time point (48%) while the other two cell lines showed a marginal response (<10%) or a decrease in FDG signal (H460 -10% decrease from control) at the early time point.

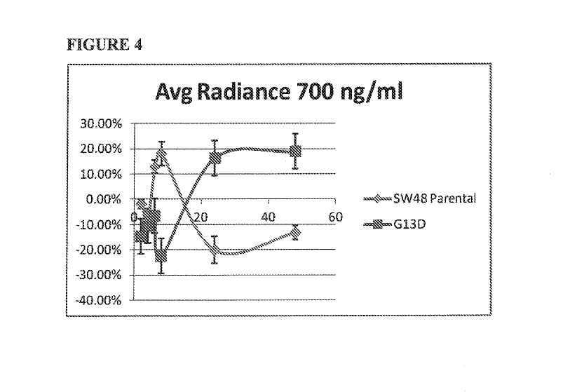

An OTOC's experiment was then conducted with the isogenic line SW48 (colon tumor) and SW48 G13D (KRAS mutant) at the 700 ng/ml concentration, with monitoring of ^{18F}-FDG uptake over time (FIG. 4). The KRAS mutant cell line behaved markedly different than those of the WT. The KRAS mutant had lower FDG uptake within an 8 hour period where the WT showed an increase in FDG uptake within the same 8 hour period. FIG. 4 shows the data plotted as normalized to control for average radiance over 60h of monitoring.

In vitro experiments with the OTOC' s suggested a difference in glucose uptake/utilization between the KRAS mutant and the WT cell line.

### Example 4

### Sensitivity of Calu-6 μOTOCs to Various Compounds

Calu-6 cells, a human lung adenocarcinoma cell line, were cultured using the 3D cell culture system of the invention to generate μtOTOCs using the following methods: μOTOCs were seeded (Day 0) at 5000 cells in Matrigel per μOTOC using the methods described in Example 1. After allowing the Matrigel to congeal at 37° C., the μOTOCs were collected and washed with PBS, polled in a T75 flask with enough media to allow oil to float overnight. Media was changed on Day 1 to aspirate the remaining oil. Examination of the OTOCs under a microscope (at 2× and 10× objectives) 3 days after seeding confirmed that the cells were evenly distributed throughout the resulting sphere, rather than layered on top of each other, as seen with other 3D cell culture methods known in the art.

On Day 3, the μOTOCs were transferred in 150 μl to 96 well ULA plates using a wide boar tip (1 μOTOC/well). Test compounds were also added (50 μl for 200 μl total) on Day 3. Each well in the 96-well plate received a different treatment. Serial dilutions of a UAE inhibitor (0-2 μM), a proteosome inhibitor (0-0.2 μM), and a VPS34 inhibitor (0-10 μM) were tested.

On Day 7 (4 days post drug treatment) μOTOCs were lysed in Cell Titer Glo by removing 100 μl of media and adding 100 μl of the reagent, incubated at 37° C. for at least 20 minutes then ATP was measured in a plate reader. IVIS Spectrum was run on Auto exposure for each plate and the Living Image analysis program was used to determine Radiance/per well. Lanes were added up and group average and error was determined.

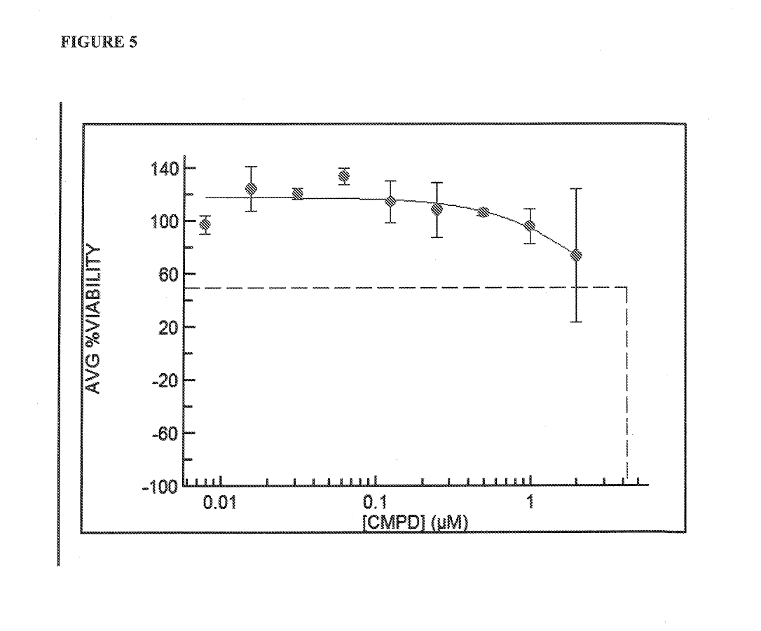

As shown in FIG. 5, the Calu-6 OTOCs were not sensitive to the UAE inhibitor tested, having an LD₅₀>2. These results are similar to the effect of this UAE inhibitor in vivo. This UAE inhibitor had no significant effect when tested in tumor xenograft models run with the Calu-6 cell line. In contrast, Calu-6 wells were very sensitive to this UAE inhibitor when grown in traditional 2D cell culture (2D LD₅₀=0.79 μM). Thus, the Calu-6 OTOCs behaved similarly to the in vivo model, whereas the opposite results were achieved from the 2D in vitro model, evidencing that the OTOC model is more predictive of the behavior Calu-6 cells in vivo than the 2D model.

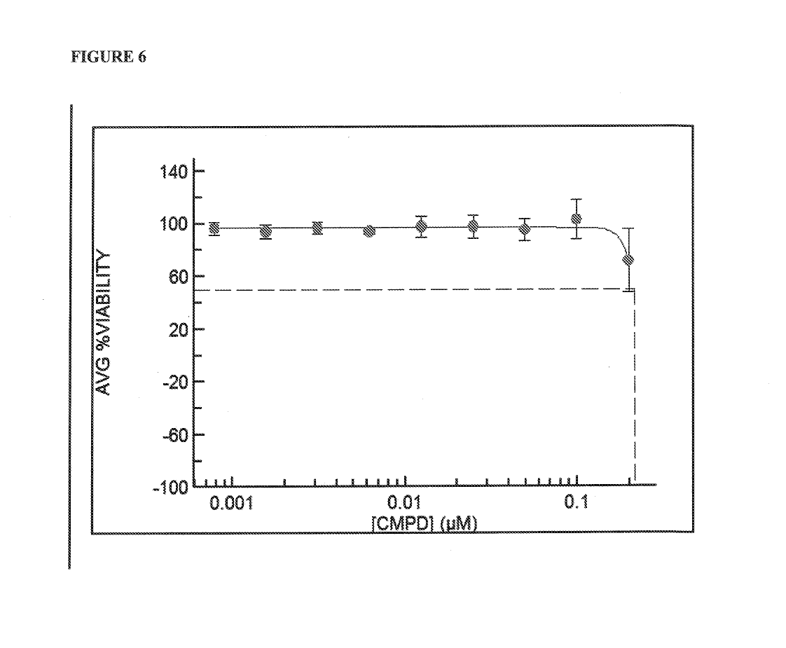

As shown in FIG. 6, the Calu-6 OTOCs were not sensitive to the proteosome inhibitor at the concentration range tested (0-0.2 μM). Since the top dose was too low, a 3D LD₅₀ for the OTOC model could not be established. Similarly, this proteosome inhibitor had no significant effect against Calu-6 cells in vivo. However, Calu-6 cells were very sensitive to this proteosome inhibitor when tested in traditional 2D cell culture, having an LD₅₀ well below the top dose tested in the OTOC model (2D LD₅₀=0.0552 μM). Again, the Calu-6 OTOCs behaved similarly to the in vivo model, whereas the opposite results were achieved from the 2D in vitro model, evidencing that the OTOC model is more predictive of the behavior Calu-6 cells in vivo than the 2D model.

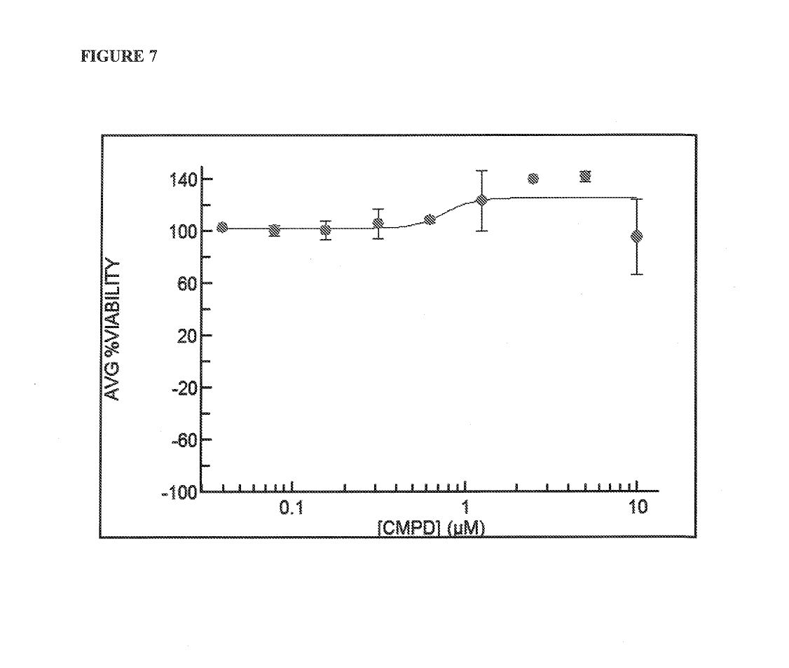

As shown in FIG. 7, the Calu-6 OTOCs were not sensitive to the VPS34 inhibitor tested, having an LD₅₀>10 μM. These results are similar to the effect of this UAE inhibitor in vivo where it was shown that this VPS34 inhibitor had no significant effect when tested in tumor xenograft models run with the Calu-6 cell line. Likewise, Calu-6 wells were not sensitive to this VPS34 inhibitor when grown in traditional 2D cell culture (2D LD₅₀=12-17 μM). Thus, the Calu-6 OTOCs behaved similarly against the VPS34 inhibitor as both in vivo and traditional in vitro models of this cell line.

### Example 5

### Proliferation Staining of OTOCs

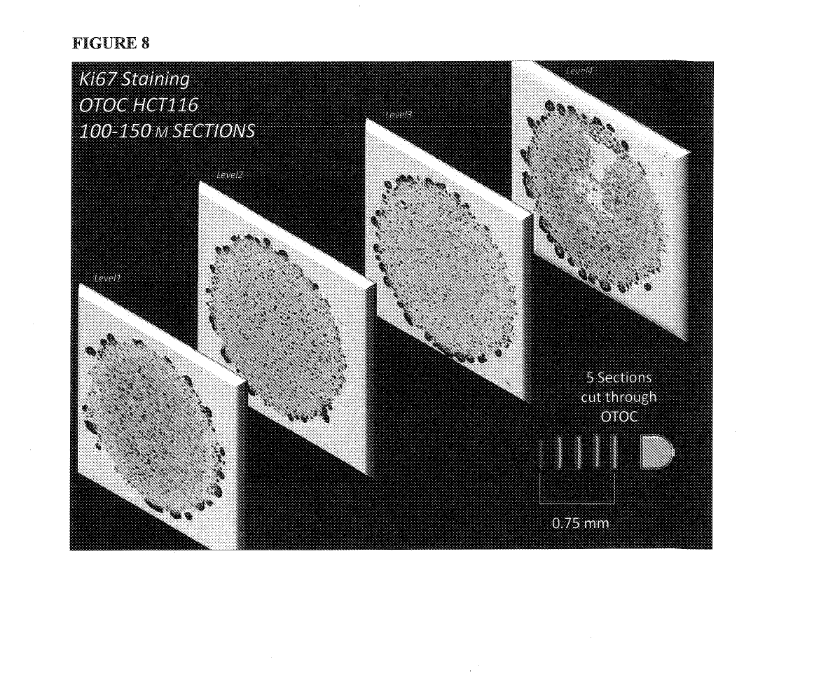

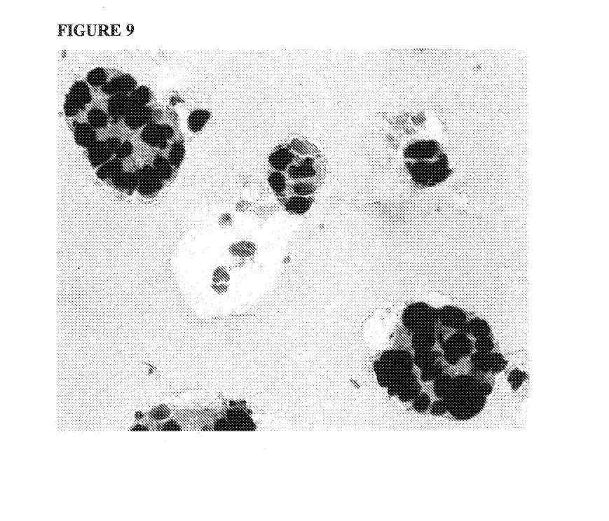

HCT-116 OTOCs were seeded/created using the methods described in Example 1 and cultured in a T225 flask. On Day 21 after the OTOCs were seeded (i.e., formed in mineral oil, isolated, washed and placed into culture), the OTOCs were fixed in formalin and embedded in Histogel, and cut into 100-150 micrometer sections cut every 0.75 mm and stained with Ki67. Images of 4 different levels of the sagittal sections are shown in FIG. 8, and FIG. 9 shows a magnified view (20×) of a portion of one of the sections shown in FIG. 8, demonstrating that the cells are proliferating (in a manner similar to clonal expansion) and evenly distributed throughout the OTOC. No layering, clumping of cells or necrosis was seen.

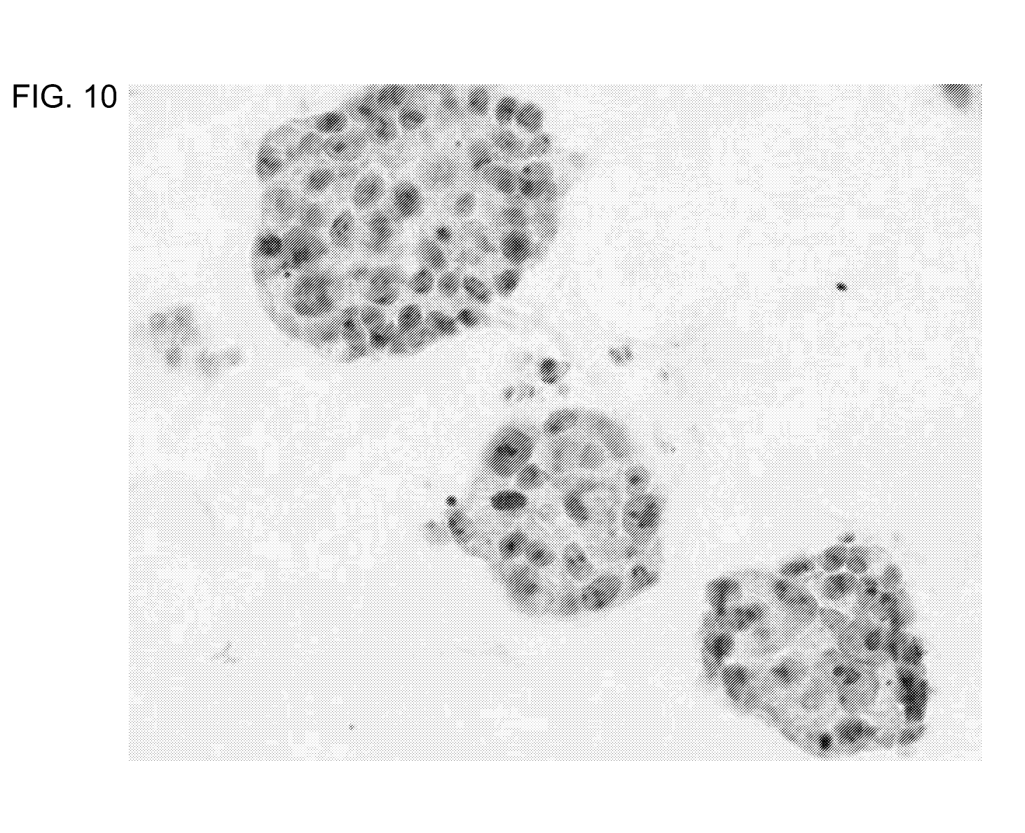

The sagittal sections were also stained with Hif1 alpha to measure hypoxia. As shown in FIG. 10, no hypoxia was seen, indicating that the OTOCs have a very nurturing growth environment.

### Example 6

### Imaging of ROTOCs

Cobalt chloride (CoCl₂) is known to cause an increase in 18F-FDG uptake in cells treated /over 24 hours. As such, 250 μM CoCl₂ was used to test whether μTOCs could be imaged.

μOTOCs of H1048 (small cell lung carcinoma) and T47D (mammary gland; ductal carcinoma) cells were generated using the method described in Example 1.

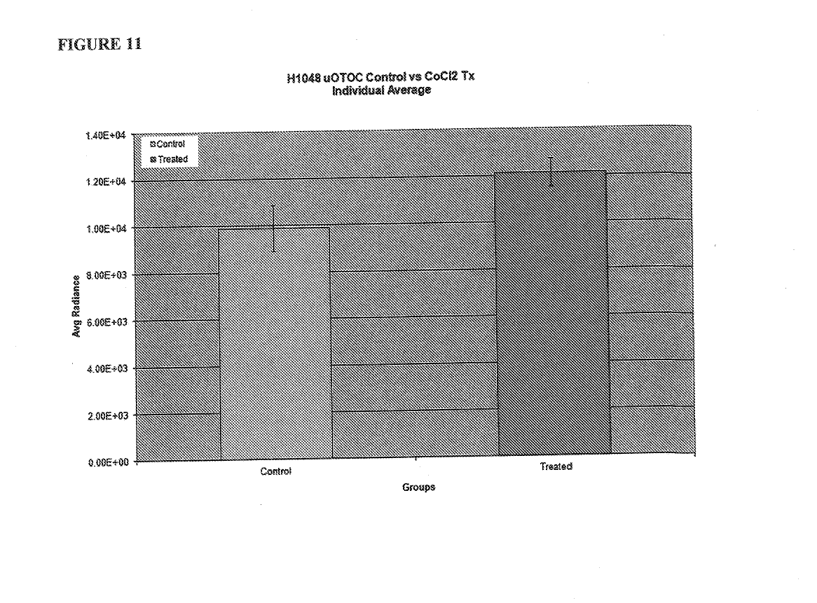

On Day 3 post seeding/creation of the OTOCs (i.e, after forming the μOTOCs in oil then transferring to T75 flask for 3 days of culture), the μOTOCs were transferred one at a time via pipette into a 96 well plate (1 μOTOC/well). 250 μM CoCl₂ was added to each well. Cerenkov Luminescence Imaging was used to monitor the FDG response in vitro. The results are shown in FIGS. 11-13.

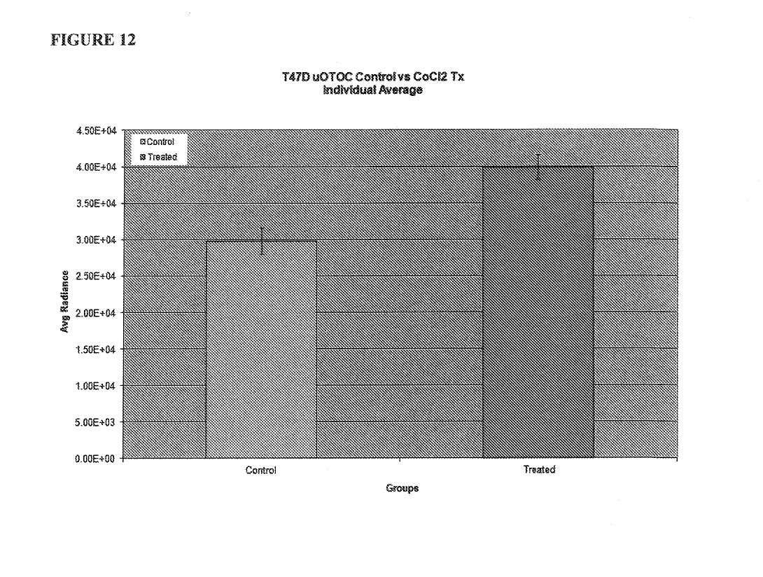

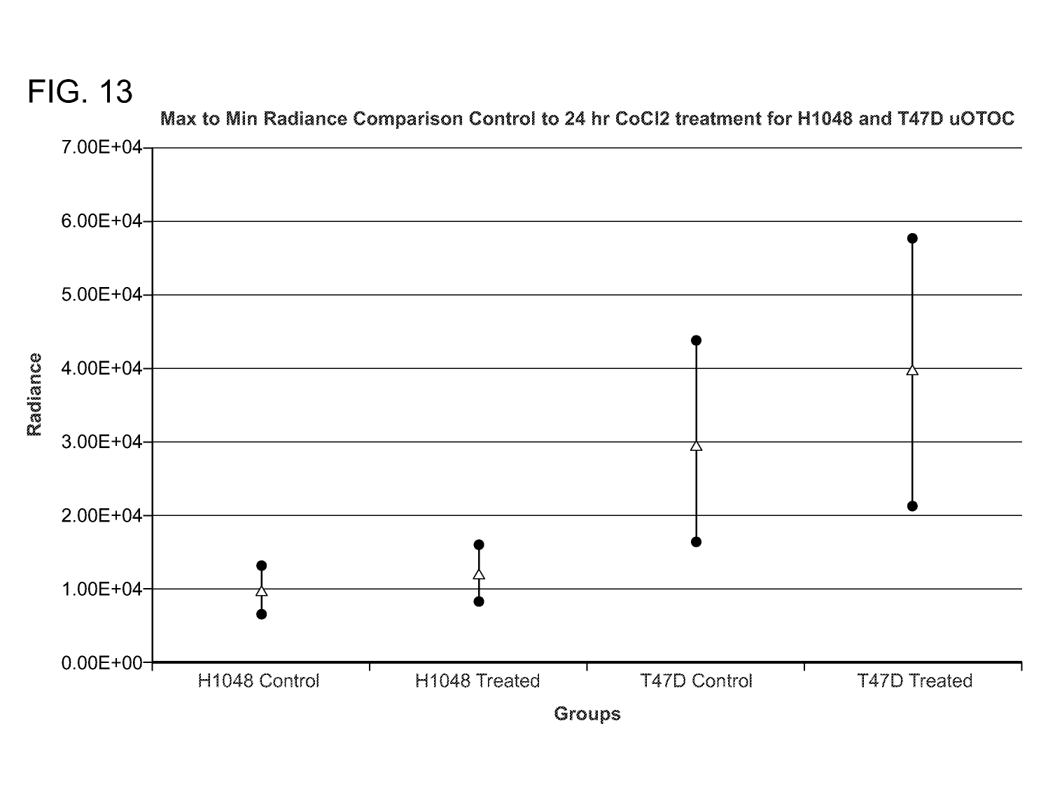

As shown in FIGS. 11 and 12, there was a 22.6% increase in 18F-FDG uptake in the H1048 μOTOCs (p value=0.926), and a 33.7% increase in FDG uptake in T47D μOTOCs (p value=0.0012), respectively. FIG. 13 shows the Max to minimum radiance comparsions control to 24 hour CoCl₂ treatment for H1048 and T47D μOTOCs. CoCl₂ is known to affect different cells differently. While it had a significant effect on the FDG uptake of the T47D μOTOCs, it did not have CoCl₂ a significant effect on the H1048 μOTOCs. Regardless of whether the effect was statistically significant, FDG uptake could still be detected, evidencing that μOTOCs are suitable for use in imaging applications.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

### References:

1. Patocs A, et al. (2007) Breast-cancer stromal cells with TP53 mutations and nodal metastases. N Engl J Med 357:2543-2551.
2. Orimo A, Weinberg R A (2006) Stromal fibroblasts in cancer: A novel tumor-promoting cell type. Cell Cycle 5:1597-1601.
3. Peehl DM (2005) Primary cell cultures as models of prostate cancer development. Endocr Relat Cancer 12:19-47.
4. Birgersdotter, A.; Sandberg, R.; Ernberg, I. Gene Expression Perturbation In vitro: A Growing Case for Three-Dimensional (3D) Culture Systems. Semin. Cancer Biol. 2005, 15, 405-412.
5. Pampaloni, F.; Reynaud, E. G.; Stelzer, E. H. The Third Dimension Bridges the Gap between Cell Culture and Live Tissue. Nat. Rev. Mol. Cell. Biol. 2007, 8, 839-845.
6. Sutherland R. M. et al (1970) Int. J. Radiat. Biol. Relat. Stud. Phys. Chem. Med.18, 491-495. Zhang X., et al. (2005) Biotechnol.Prog., 21, 1289-1296.
7. Hirschhaeuser et al. Multicellular tumor spheroids: An underestimated tool is catching up again. 2010, J. Biotechnol. 148(1): 3-15.
8. Smith BH et al. Hydrophilic agarose macrobead cultures select for outgrowth of carcinoma cell populations that can restrict tumor growth. Cancer Res. 2011 Febm 1; 71(3):725-35.
9. Zhang, X.; Wang, W.; Yu, W.; Xie, Y.; Zhang, Y.; Ma, X. Development of an In vitro Multicellular Tumor Spheroid Model Using Microencapsulation and Its Application in Anticancer Drug Screening and Testing. Biotechnol Prog. 2005, 21, 1289-1296.
10. Marc A., Markvicheva E., Jourdain C., Bezdetnaya L., Merlin J. L., Guillemin F., Zubov V., Goergen J.L. (2005). Spheroids formation by encapsulation of cancer cells to mimic small size tumors, In : Animal Cell Technology meets Genomics. Godia F. and Fussenger M. (Eds.), Springer, Netherlands , 261-263.
11. Markvicheva E., Bezdetnaya L., Bartkowiak A., Marc A., Goergen J. L., Guillemin F., Poncelet D. (2003). Encapsulated multicellular tumor spheroids as a novel in vitro model to study small size tumors. Hemijska industrija 57 (12), 585-588
12. Biggs et al. Antitumor Compound Testing in Gioblastoma Organotypic Brain Cultures. J Biomol Screen September 2011 vol. 16 no. 8 805-817.
13. Vaira et al., Preclinical model of organotypic culture for pharmacodynamic profiling of human tumors. PNAS 2010, 107(18), 8352-8356.
14. Mazzoleni et al. Modeling Tissues in 3D: the next future of pharmaco-toxicology and food research? Genes Nutr, 2009 4: 13-22.
15. Friedrich, J.; Seidel, C.; Ebner, R.; Kunz-Schughart, L. A. Spheroid-Based Drug Screen: Considerations and Practical Approach. Nat. Protoc. 2009, 4, 309-324.
16. Herrmann, R.; Fayad, W.; Schwarz, S.; Berndtsson, M.; Linder, S. Screening for Compounds That Induce Apoptosis of Cancer Cells Grown as Multicellular Spheroids. J. Biomol. Screen. 2008, 13, 1-8.
17. Kunz-Schughart, L. A.; Freyer, J. P.; Hofstaedter, F.; Ebner, R. The Use of 3-D Cultures for High-Throughput Screening: The Multicellular Spheroid Model. J. Biomol. Screen. 2004, 9, 273-285.
18. Li, Q.; Chen, C.; Kapadia, A.; Zhou, Q.; Harper, M. K.; Schaack, J.; LaBarbera, D. V. 3D Models of Epithelial-Mesenchymal Transition in Breast Cancer Metastasis: High-Throughput Screening Assay Development, Validation, and Pilot Screen. J. Biomol. Screen. 2011, 16, 141-154.
19. Moritz, W. C.; Kelm, J. C.; Clavien, P.-A. C.; Hoerstrup, S. P. C. Hanging Drop Plate, 2010, WO2010/031194. International Application No. PCT/CH2008/00391.
20. Nyga, Agata, Umber Cheema, and Marilena Loizidou. “3D Tumour Models: Novel in vitro Approaches to Cancer Studies.” Journal of Cell Communication and Signaling 5, no. 3 (August 2011): 239-248.
21. Haycock, J W. “3D Cell Culture: A Review of Current Approaches and Techniques.” Methods Mol Biol 695 (2011): 1-15.
22. Burdick, Jason A, and Gordana Vunjak-Novakovic. “Engineered Microenvironments for Controlled Stem Cell Differentiation.” Tissue Eng Part A.; 15(2) (February 2009): 205-219.
23. Baker, Brandon M, and Chen, Christopher S. “Deconstructing the Third Dimension-How 3D cell culture Microenvironments Alter Cellular Cues.” Journal of Cell Science 125 (2012); 1-10.
24. International Application Publication No. WO2011161172
25. International Application Publication No. WO2006136953
26. U.S. Pat. No. 5,643,569, Jain
27. U.S. Pat. 4,352,883, Lim
28. Gill, BBartley J., Gibbons, Don L., Roudsari, Laila C., et al., “A synthetic Matrix with Independently Tunable Biochemistry and Mechanical Properties to Study Epithelial Morphogenesis and EMT in a Lung Adenocarcinoma Model.” Cancer Res., 2012; 72:6013-6023. Published Online First Sep. 4, 2012.

1. An isolated, unencapsulated, three-dimensional, cell culture product comprising a naturally-derived cell matrix distributed throughout the product and having dimensions ranging from about 1-10 mm in diameter (length), and about 1-5 mm in width (thickness).

2. The cell product of claim 1, wherein the dimensions comprise a diameter ranging from 5-8 mm and a width ranging from about 3-4.5 mm, preferably a diameter ranging from 1.5-1.8 mm, and a width ranging from about 1.0-1.4 mm.

3. (canceled)

4. A vessel comprising a plurality of the cell culture products of claim 1 and a medium suitable for suitable for supporting growth of the cell product.

5. A method for producing an isolated, unencapsulated, three-dimensional oganotypic cell culture product, the method comprising the steps of
a. harvesting one or more cells from an in vitro culture;
b. resuspending the one or more cells with a naturally derived gel matrix under conditions sufficient to form a liquid cell suspension;
c. dispensing at least a portion of the liquid cell suspension directly into a hydrophobic solution under conditions sufficient to enable the liquid cell suspension to form a gelled three-dimensional cell matrix within the hydrophobic solution;
d. isolating the three-dimensional cell matrix from the hydrophobic solution; and
e. culturing the three-dimensional cell matrix in a growth medium under conditions sufficient for promoting proliferation of the cells within the three-dimensional cell matrix, thereby producing an unencapsulated, three-dimensional organotypic culture.

6. The method of claim 5, wherein the naturally derived gel matrix comprises a sol-gel matrix, a solubilized basement membrane preparation, or both.

7. (canceled)

8. (canceled)

9. The method of claim 5, wherein the naturally derived gel matrix comprises one or more matrix proteins selected from the group consisting of laminin, collagen IV, heparin sulfate proteoglycans, and enactin, nidogen, or any combination thereof.

10. The method of claim 5, wherein the naturally derived gel matrix further comprises one or more growth factors selected from the group consisting of TGF-beta, epidermal growth factor (EGF), insulin-like growth factor (IGF-1), fibroblast growth factor (FGF), tissue plasminogen activator, 3 4(tPA), nerve groth factor (NGF), wth atelet-derived growth factor (PDGF), or any combination thereof.

11. (canceled)

12. The method of claim 5, wherein the naturally derived gel matrix further comprises heparin sulfate proteoglycan (perlecan), one or more matrix metalloproteinases, or both.

13. (canceled)

14. The method of claim 5, wherein the naturally derived gel matrix is Matrigel.

15. The method of claim 5, wherein the hydrophobic solution is mineral oil.

16. (canceled)

17. The method of claim 5, wherein the conditions sufficient for forming a liquid suspension comprise cooling the gel matrix to approximately 4° C. prior to resuspending the one or more cells in the gel matrix, and the conditions sufficient to form a gelled three-dimensional cell matrix comprise dispensing the liquid cell suspension into a hydrophobic solution that has a temperature of 20-25° C.

18. (canceled)

19. The method of claim 5, wherein the three-dimensional culture environment comprises a vessel selected from the group consisting of a cell culture flask, a petri dish or a multi-well plate.

20. (canceled)

21. The method of claim 5, wherein the one or more cells are tumor cells, stem cells, blood cells, immune cells, or inflammatory cells.

22. The method of claim 21, wherein the stem cells are embryonic stem cells, adult stem cells or induced pluripotent stem cells; and the tumor cells are derived from a tumor type selected from the group consisting of: a hematologic tumor, a lymphoma, a lung tumor, a prostate tumor, a breast tumor, an ovarian tumor, a cervical tumor, a colon tumor, a gastric tumor, a pancreatic tumor and a melanoma.

23. (canceled)

24. An in vitro method for assessing the pharmacological response of cells to a therapeutic agent, the method comprising the steps of
a. producing an unencapsulated, three-dimensional organotypic culture according to the method of claim 5;
b. contacting the unencapsulated, three-dimensional organotypic culture with a therapeutic or cytotoxic agent; and
c. measuring one or more characteristics of the unencapsulated, three-dimensional organtoypic culture subsequent to the contact with the therapeutic or cytotoxic agent.

25. The method of claim 24, wherein the cells are selected from the group consisting of tumor cells, stem cells, blood cells, immune cells, and inflammatory cells.

26. The method of claim 25, wherein the stem cells are embryonic stem cells, adult stem cells or induced pluripotent stem cells; and the tumor cells are derived from a tumor type selected from the group consisting of: a hematologic tumor, a lymphoma, a lung tumor, a prostate tumor, a breast tumor, an ovarian tumor, a cervical tumor, a colon tumor, a gastric tumor, a pancreatic tumor, and a melanoma.

27. (canceled)

28. The method of claim 24, wherein the one or more characteristics are selected from the group consisting of RNA expression, DNA expression, protein expression, cellular uptake, cellular signaling, cell viability, apoptosis, cell shedding, cellular necrosis, cellular heterogeneity, multicellular interactions, and sensitivity to the therapeutic agent, or any combination thereof.

29. The method of claim 24, wherein the therapeutic or cytotoxic agent comprises a detectable label selected from a fluorescent label or a radiolabel.

30. (canceled)

31. The method of claim 24, wherein the measuring step comprises imaging of the unencapsulated, three-dimensional organotypic culture using a technology selected from the group consisting of optical imaging, nuclear imaging, MRI, SPECT, PET, and CLI.

32. (canceled)

33. The method of claim 24, wherein the measuring step comprises an in vitro technique selected from the group consisting of: immunoflourescence, immunohistochemistry, western blotting, northern blotting and southern blotting, a proliferation assay, a cell viability assay, an apoptosis assay, an internalization assay, a cell penetration assay, or any combination thereof.

34. (canceled)

35. An in vitro method for assessing the response of cells to one or more environmental conditions, the method comprising the steps of:
a. producing an unencapsulated, three-dimensional organotypic cell culture according to the method of claim 5;
b. obtaining a baseline measurement of one or more characteristics of the unencapsulated organotypic culture;
c. modulating one or more culture conditions for a prolonged period of time; and
d. obtaining a second measurement of the one or more characteristics of the three-dimensional cell culture subsequent to modulation of the one or more culture conditions;
e. comparing the second measurement of the one or more characteristics to the baseline measurement(s); wherein in a change in the one or more characteristics is indicative of the response of the cells to the modulated culture condition.

36. The method of claim 35, wherein the one or more characteristics are selected from the group consisting of: RNA expression, DNA expression, protein expression, cellular uptake, cellular signaling, cell viability, apoptosis, cell shedding, cellular necrosis, cellular heterogeneity, and multicellular interactions, or any combination thereof.

37. The method of claim 35, wherein the modulation step comprises modulation of a culture condition selected from the group consisting of: oxygen level, nitrogen level, carbon dioxide level, temperature, growth media, a growth media supplement, pH, and length of culture.

38. The method of claim 35, wherein the measuring step comprises imaging of the unencapsulated organotypic culture using a technique selected from the group consisting of optical imaging, nuclear imaging, MR1, SPECT, PET, and CLI.

39. (canceled)

40. The method of claim 35, wherein the measuring step comprises an in vitro technique selected from the group consisting of: immunoflourescence, immunohistochemistry, western blotting, northern blotting and southern blotting, a proliferation assay, a cell viability assay., an apoptosis assay, an internalization assay, and a cell penetration assay, or any combination thereof.

41. (canceled)
